(19) United States
(12) Patent Application Publication
(10) Pub. No.: US 2025/0090865 A1
(43) Pub. Date: Mar. 20, 2025

(54) LESIONAL DOSIMETRY FOR TARGETED RADIOTHERAPY OF CANCER

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Steven M. LARSON, New York, NY (US); Audrey MAUGUEN, New York, NY (US); John L. HUMM, New York, NY (US); Ravinder K. GREWAL, New York, NY (US); Alan L. HO, New York, NY (US)

(21) Appl. No.: 18/562,263

(22) PCT Filed: May 26, 2022
(86) PCT No.: PCT/US2022/031066

§ 371 Date: Nov. 17, 2023

Related U.S. Application Data
(60) Provisional application No. 63/193,700, filed on May 27, 2021.

(51) Int. Cl. A61N 5/10; G06T 7/00; G16H 20/40

(52) U.S. Cl. CPC A61N 5/1039; A61N 5/1031; G06T 7/0012; G16H 20/40; A61N 2005/1021; G06T 2207/10104; G06T 2207/10108; G06T 2207/30096

(57) ABSTRACT

The present disclosure provides lesional dosimetry methods for predicting the radioisotope activity required to deliver a therapeutic dose of radiation to induce an anti-tumor response in tumor lesions in a subject in need thereof. Specifically implemented is a computer-implemented dosimetry method comprising: detecting, in a single-time-point medical image of a patient, for each cancerous lesion of the patient, an indicator of a surrogate of a radiotherapeutic compound. The medical images captured within a predetermined time range following administration of the surrogate to the patient. Using each indicator, determining an uptake metric corresponding to uptake of the surrogate by each corresponding cancerous lesion. Generating, based on the predetermined time range and the uptake metric, using a dosimetry biomarker based on uptake of the surrogate by subjects in a cohort of subjects, a radiation dose prediction for administration of the radiotherapeutic compound to the patient. Resulting in providing the radiation dose prediction for subsequent use by a healthcare professional in determining a treatment protocol for treating the cancerous lesions of the patient using the radiotherapeutic compound.

### CROSS-REFERENCE TO RELATED APPLICATIONS

This application claims the benefit of and priority to U.S. Provisional Patent Application No. 63/193,700, filed May 27, 2021, the entire contents of which are incorporated herein by reference.

### TECHNICAL FIELD

The present technology relates generally to a lesional dosimetry approach for predicting the radioisotope activity required to deliver a therapeutic dose of radiation to induce an anti-tumor response in tumor lesions in a patient, and generating a biomarker for such predictions.

### BACKGROUND

The following description of the background of the present technology is provided simply as an aid in understanding the present technology and is not admitted to describe or constitute prior art to the present technology.

Precision medicine involves tailoring or optimizing treatment for a unique cancer in individual patients. For patients with differentiated thyroid cancer (DTC), for example, distant metastases are detected in 10-15% at some point in the course of their disease. For advanced thyroid cancer, treatment of metastatic DTC (mDTC) with radioiodine ¹³¹I therapy (RAI) has been life-saving for many patients. However, not all patients benefit from RAI and side effects can be serious and, in some cases, life-threatening.

Response of cancer (e.g., thyroid cancer) to RAI is radiation dose-related, but unlike modern external beam radiotherapy, there are no dosimetry methods to reliably predict which patients with metastatic cancer (e.g., metastatic thyroid cancer) are likely to respond to RAI therapy. Accordingly, many patients continue to receive multiple empirical therapeutic doses of radiotherapy (e.g., with ¹³¹I or otherwise) that may be ineffective and that could cause considerable morbidity, with potential toxicity to, for example, the bone marrow, lung, salivary glands, and/or other tissues.

Accordingly, there is an urgent need for lesional dosimetry methods that can accurately and reliably predict which patients with metastatic cancer (e.g., metastatic thyroid cancer) are likely to respond to radiotherapy.

### SUMMARY

In one aspect, various embodiments of the present disclosure relate to a computer-implemented dosimetry method. The dosimetry method may comprise detecting, for each cancerous lesion of a patient, an indicator of a surrogate of a radiotherapeutic compound. The indicator may be detected in one or more medical images of the patient. The one or more medical images of the patient may be a single medical image captured at a single time point. The one or more medical images may be captured within a predetermined time range following administration of the surrogate to the patient. The method may comprise determining an uptake metric corresponding to uptake of the surrogate by each corresponding cancerous lesion. The uptake metric may be determined based on each detected indicator. The method may comprise generating, based on the predetermined time range and the uptake metric, a radiation dose prediction for administration of the radiotherapeutic compound to the patient. The radiation dose prediction may be generated using a dosimetry biomarker/signature that is based on uptake of the surrogate by subjects in a cohort of subjects. The method may comprise providing the radiation dose prediction for subsequent use in determining a treatment protocol for treating the cancerous lesions of the patient using the radiotherapeutic compound. The radiation dose prediction may be used by a healthcare professional, such as a physician, in determining the treatment protocol. The radiation dose may be provided to a user such as the healthcare professional, or to an healthcare facility or otherwise another entity through which the radiation dose may be accessible.

In various embodiments, the uptake metric may be based on a standardized uptake value (SUV). The uptake metric may be the SUV normalized by lean body mass (SUL).

In various embodiments, the medical image may be captured using a medical imaging system that is, or that comprises, a positron emission tomography (PET) scanner. The medical image may be, may comprise, or may be based at least in part on a PET scan. Alternatively or additionally, the medical imaging system may be, or may comprise, a single photon emission computed tomography (SPECT) scanner. Alternatively or additionally, the medical image may be, may comprise, or may be based at least in part on a SPECT scan.

In various embodiments, the surrogate may be an isotope of the radiotherapeutic compound. The surrogate may be, or may comprise, for example, Iodine-124, and correspondingly, the radiotherapeutic compound may be, or may comprise, for example, Iodine-131.

In various embodiments, the biomarker indicates dosages corresponding to different uptake metrics.

In various embodiments, the method may comprise generating the biomarker. Generating the biomarker may comprise, for each subject in the cohort of subjects, obtaining a plurality of images captured at a plurality of times following administration of the surrogate. Each subject of the cohort of subjects may have one or more cancerous lesions. The cancerous lesions may be of the same type or otherwise shares characteristics with the cancerous lesion of the patient. The method may comprise detecting, in the plurality of images, for each cancerous lesion of each subject, a surrogate indicator corresponding to presence of the surrogate in each cancerous lesion. The method may comprise determining, based on the surrogate indicators for the subjects in the cohort of subjects, one or more surrogate uptake metrics indicative of uptake of the surrogate by the corresponding cancerous lesions of the subjects. The method may comprise performing operations on the uptake metrics to generate the biomarker for predicting radiation dose for the radiotherapeutic compound. The operations may comprise application of a model, such as a generalized estimating equation (GEE) model or another estimating equation model, to the uptake metrics.

In various embodiments, the plurality of times may comprise four times following administration of the surrogate. In other embodiments, the plurality of times may comprise three times following administration of the surrogate. In other embodiments, the plurality of images may comprise five or more times following administration of the surrogate. In certain embodiments, the plurality of images may differ for each subject in the cohort of subjects, such that a subset of subjects have one or more images captured at four times following administration of the surrogate to each subject in the subset, and another subset of subjects have one or more images captured at a number greater than or less than four times following administration of the surrogate to each subject in the another subset of subjects.

In various embodiments, the operations may comprise generating areas under the curve (AUCs) based on the surrogate uptake metrics.

In various embodiments, the radiotherapeutic compound may be I-131, PSMA-617, lutetium Lu 177 dotatate, or radiolabeled DOTA hapten.

In another aspect, the various embodiments of the present disclosure relate to a computer-implemented method for generating a biomarker. The method may comprise, for each subject in a cohort of subjects, obtaining a plurality of images captured using a medical imaging system at a plurality of times following administration of at least one of (i) a radiotherapeutic compound, or (ii) a surrogate for the radiotherapeutic compound. Each subject may have one or more cancerous lesions. The method may comprise detecting, in the plurality of images, for each cancerous lesion of each subject, a surrogate indicator corresponding to presence of the surrogate in each cancerous lesion. The method may comprise determining, based on the surrogate indicators, one or more surrogate uptake metrics indicative of uptake of the surrogate by the corresponding cancerous lesions. The method may comprise performing operations on the uptake metrics to generate a biomarker for predicting radiation dose for the radiotherapeutic compound. The operations may comprise application of an estimation model to the uptake metrics. The method may comprise providing the biomarker for subsequent prediction of radiation dose in determining treatment protocols for patients with one or more cancerous lesions based on uptake metric and amount of time following administration of the surrogate to the patient.

In various embodiments, the plurality of images are captured following administration of only the surrogate of the radiotherapeutic compound to each subject and not the radiotherapeutic compound. The plurality of images may be captured following administration of the surrogate to a subset of the subjects, and administration of the radiotherapeutic compound to another subset of the subjects. The plurality of images may be captured following administration of only the radiotherapeutic compound to each subject.

In various embodiments, the estimation model is based on a generalized estimating equation (GEE).

In various embodiments, the plurality of times comprises four times following administration of the surrogate. In other embodiments, the plurality of times may be another number of times, such as three, five, or greater than five.

In various embodiments, the operations further comprise generating areas under the curve (AUCs) based on the surrogate uptake metrics.

In various embodiments, the medical imaging system may be, or may comprise, a positron emission tomography (PET) scanner, and the medical image may be, may comprise, or may be based at least in part on a PET scan. Alternatively or additionally, the medical imaging system may be, or may comprise, a single photon emission computed tomography (SPECT) scanner, and the medical image may be, may comprise, or may be based at least in part on a SPECT scan.

In various embodiments, the surrogate may be, or may comprise, an isotope of the radiotherapeutic compound. For example, the surrogate may be or may comprise Iodine-124, and the radiotherapeutic compound may be or may comprise Iodine-131.

In various embodiments, the biomarker may indicate dosages corresponding to different uptake metrics.

In various embodiments, the method may comprise applying the generated biomarker to one or more patients. The method may comprise detecting, for each cancerous lesion of the patient, an indicator of a surrogate of a radiotherapeutic compound. The medical image may be a single-time-point medical image (e.g., one or more medical images captured at one visit of the patient to an imaging facility with the medical imaging system). The single-time-point medical image may be captured within a predetermined time range following administration of the surrogate to the patient (e.g., the one visit to the imaging facility may be no earlier than a first time threshold after administration of the surrogate to the patient, but no later than a second time threshold after administration of the surrogate to the patient). The method may comprise determining, based on each indicator, an uptake metric corresponding to uptake of the surrogate by each corresponding cancerous lesion. The method may comprise generating, based on the uptake metric and the predetermined time range, using the biomarker generated based on uptake metrics measured following administration of the surrogate to the cohort of subjects, a prediction of radiation dose for administration of the radiotherapeutic compound to the patient. The method may comprise providing the radiation dose prediction for subsequent use in determining a treatment protocol for treating the cancerous lesions of the patient using the radiotherapeutic compound. Providing the radiation dose prediction may comprise at least one of storing the radiation dose prediction in a non-transitory computer-readable storage medium, outputting the radiation dose prediction using, for example, a display screen or a printer, or transmitting the radiation dose prediction to another computing device or computing system (e.g., over the internet and/or another telecommunication network, via wired and/or wireless communication protocols). The subsequent use of the radiation dose prediction may be by a healthcare professional (such as a radiologist or other physician, or a medical technician), and providing the radiation dose prediction may comprise making the radiation dose prediction accessible to the healthcare professional (e.g., via a software application, via the internet or other network, via an output device such as a display screen, etc.).

In various embodiments, the uptake metric may be based on a standardized uptake value (SUV). The uptake metric may be the SUV normalized by lean body mass (SUL).

In various embodiments, the radiotherapeutic compound may be or may comprise I-131, PSMA-617, lutetium Lu 177 dotatate, and/or radiolabeled DOTA hapten.

In yet another aspect, various embodiments relate to a computer-implemented method. The method may comprise acquiring a medical image of a patient captured at an amount of time following administration of a surrogate of a radiotherapeutic compound to the patient. The amount of time may fall within a predetermined time range following the administration of the surrogate. The method may comprise determining, based on the medical image, an uptake metric corresponding to uptake of the surrogate by each cancerous lesion of the patient. The method may comprise generating, based on the amount of time and the uptake metrics, using a biomarker, a radiation dose prediction for administration of the radiotherapeutic compound to the patient. The radiation dose may be predicted to produce an anti-tumor response. The biomarker may be based on cohort uptake metrics measured in multiple-time-point medical images of each subject in a cohort of subjects following administration of the surrogate or administration of the radiotherapeutic compound to each subject in the cohort. The method may comprise outputting the radiation dose for use in determining a treatment protocol comprising using the radiotherapeutic compound to treat the cancerous lesions of the patient based on the radiation dose prediction.

In various embodiments, the method may comprise capturing the medical image using a medical imaging system.

In various embodiments, the method may comprise administering the surrogate of the radiotherapeutic compound to the patient. The surrogate may be administered the amount of time prior to acquiring the medical image.

In various embodiments, determining the uptake metric corresponding to uptake of the surrogate by each cancerous lesion of the patient may comprise detecting, in the medical image, for each cancerous lesion of the patient, an indicator of the surrogate, and generating the uptake metric based on the indicator.

In various embodiments, the method may comprise administering the treatment to the patient.

In yet another aspect, various embodiments relate to a method that may comprise: capturing, using a medical imaging system, a medical image of a patient at an amount of time following administration of a surrogate of a radiotherapeutic compound to the patient, the amount of time falling within a predetermined time range following the administration of the surrogate; determining, based on the medical image, an uptake metric corresponding to uptake of the surrogate by each cancerous lesion of the patient; generating, based on the single time point and the uptake metric, using a biomarker generated based on uptake metrics measured following administration of the surrogate or administration of the radiotherapeutic compound to a cohort of subjects, a prediction of radiation dose for administration of the radiotherapeutic compound to the patient; and determining a treatment protocol for treating the cancerous lesions of the patient based on the prediction of radiation dose.

In various embodiments, the method may comprise administering a treatment to the patient based on the treatment protocol.

In any and all embodiments of the methods disclosed herein, the patient or patients comprises a genetic mutation that regulates uptake of the surrogate. In certain embodiments, the genetic mutation is BRAF V600E. Additionally or alternatively, in some embodiments of the methods disclosed herein, the patient or patients have received or are receiving a MEK inhibitor and/or a BRAF inhibitor. Examples of BRAF inhibitors include, but are not limited to GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436). Examples of RAF/MEK/ERK inhibitors include, but are not limited to Vemurafenib (Zelboraf) and Dabrafenib (GSK2118436), Encorafenib, TAK-632, PLX4720, MLN2480, Cobimetinib (GDC-0973), MEK 162, R05126766, GDC-0623, VTX11e, Selumetinib (AZD6244), PD0325901, Trametinib (GSK1120212), U0126-EtOH, PD184352 (CI-1040), Refametinib, PD98059, BIX02189, Binimetinib, Pimasertib (AS-703026), SL327, BIX02188, AZD8330, TAK-733, PD318088, SCH772984, and FR 180204.

In yet other aspects, various embodiments of the disclosure relate to a computing system (which may be, or may comprise, one or more computing devices) comprising one or more processors that are configured to implement any of the methods disclosed herein.

In yet other aspects, various embodiments of the disclosure relate to non-transitory computer-readable storage media comprising instructions configured to cause one or more processors of a computing system (which may be, or may comprise, one or more computing devices) to implement any of the methods disclosed herein.

### BRIEF DESCRIPTION OF THE DRAWINGS

FIG. 1 depicts an example system for implementing the disclosed dosimetry approach, according to various potential embodiments.

FIG. 2 depicts an example biometric generation and dosimetry process, according to various potential embodiments.

FIG. 3 shows a simplified block diagram of a representative server system and client computer system usable to implement certain embodiments of the present disclosure.

FIG. 4 shows an example of four ¹²⁴I PET scans conducted at 24, 48, 72, and 120 hour post-oral radioiodine administration. The clearance curves (SUVmax plotted versus time in days) for individual neck and lung lesions of size >0.5 cc is shown in the view graph.

FIG. 5 shows a learning dataset used to select best predictor as the biomarker. 48 h SUV (optimal predictor) versus AUC (dots represent patients; black line is the linear regression line while gray area is the 95% prediction interval). AUC in μCi-hr plotted against SUV at 48 hours post I¹²⁴ dose for all individual lesions (SUV >1) of metastatic thyroid cancer.

FIG. 6 shows distribution of treatment dose given in 169 treated lesions (15 patients that received I¹²⁴).

FIG. 7 shows the results of leave-one-out cross-validation (SUV analysis).

FIG. 8 shows maximum intensity projection (MIP) PET I-124 images at 48 hours of 21 patients in teaching set (FIG. 5).

FIG. 9 shows an abbreviated table of parameters determined for each lesion with measured ¹²⁴I radioiodine uptake from patient #1.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

The use of ¹²⁴I has been proposed as a theranostic solution to the problem of RAI dosimetry. In principle, accurate lesion dosimetry can be achieved using the four-day T-1/2 positron-emitting isotope ¹²⁴I and serial PET imaging using the MIRD approach (MIRD 2020). A simple correction for the physical half-life and emissions between imaging isotope ¹²⁴I and therapeutic isotope ¹³¹I provides the capability to project the lesion doses from a planned ¹³¹I therapy administration. Such radionuclide dosimetry may allow nuclear medicine physicians and endocrinologists to better identify patients likely to benefit from radioiodine and, importantly, patients who will not, which would prevent unnecessary treatments where tumor doses are below the levels necessary to achieve therapeutic responses. The major drawbacks to adopting ¹²⁴I imaging-based thyroid radionuclide dosimetry are the considerable cost and time involved in performing multiple PET scans on units typically employed as workhorses for FDG standard-of-care scans, and the inconvenience to patients who would need to return for imaging on four separate occasions.

The present disclosure provides a single-time point dosimetry method using PET/CT ¹²⁴I imaging, based on a 48-hour time point/48-hour effective half-life principle. Additionally or alternatively, SPECT imaging may be used. The present methods are based on the discovery that a single time point of quantitative PET imaging of individual lesions in cancer patients (e.g., RAI refractory/resistant thyroid cancer patients) at 48 hours could determine whether RAI treatment would be effective in treating the individual lesions. Provided herein is a validated lesional dosimetry tool that uses a single radioisotope PET imaging time point to predict the overall dosimetry to provide the prescribing physician with a lesion dose estimate for any selected administration activity. The methods of the present technology validated the 48-hour single-time point imaging as a predictor of lesion dosimetry, thus reducing the need for data acquisition to a single scan data point. The methods disclosed herein incorporates useful information about the variability in lesion uptake by considering all lesions from all subjects in the calculation of a prediction interval, in order to best determine the predicted prescribed radioactivity to achieve a radiation-absorbed lesion dose with a given precision, typically 90% or 95% probability.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the terms “lean body mass” or “LBM” refer to a part of body composition that is defined as the difference between total body weight and body fat weight. LBM counts the mass of all organs except body fat.

As used herein, “radiation-absorbed dose” refers to radiation-absorbed dose to individual lesions, or critical organs with the units of cGy, Sv, or rad.

As used herein “prescribed administered radioactivity,” in mCi or MBq or “maximum tolerated activity” (MTA) in mCi or MBq refer to amounts of radioactivity prescribed or administered.

As used herein, the terms “standardized uptake value” or “SUV” refer to a ratio of the concentration of a radiopharmaceutical in a volume of tissue in microcuries of injected agent per volume to concentration in the body of a subject if uniformly distributed (determined by a standard body phantom). SUV may be defined as the ratio of activity (of a radiopharmaceutical) per unit volume of a region of interest (ROI) to the activity per unit whole body volume. An SUV of 1.0 is achieved in any tissue volume when the count rate is equal to the count rate of the uniformly distributed activity in the body phantom. An SUV of 1.0 or higher is generally considered to be indicative of malignant tissue. As used herein, “standardized uptake value-lean” or “SUL” means the standardized uptake value (SUV) normalized by lean body mass.

As used herein, the terms “subject”, “patient”, or “individual” can be an individual organism, a vertebrate, a mammal, or a human. In some embodiments, the subject, patient or individual is a human.

As used herein, the term “surrogate” refers to a molecule that mimics several pharmacological properties of a therapeutic radiopharmaceutical such as tissue uptake, retention, catabolism, clearance etc. In some embodiments, the surrogate does not induce a therapeutic response in the subject, and may be used for quantitative imaging to predict the activity of the therapeutic radiopharmaceutical (or the responsiveness of the subject to the therapeutic radiopharmaceutical). Examples of surrogate-therapeutic radiopharmaceutical pairings include but are not limited to ¹²⁴I and ¹³¹I; ¹⁷⁷Lu and PSMA 617; ¹⁷⁷Lu and Luthethera; ⁹⁹mTc-macroaggregated albumin and ⁹⁰Y etc.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Lesional Dosimetry Methods and Systems of the Present Technology

Treatments for cancer can be ineffectual or minimally effectual at inducing an anti-tumor response in a patient, and more seriously, can harm healthy tissues as well as diseased tissues. A treatment protocol that is safe and effective for one patient will not necessarily have the same safety and effectiveness profile in another patient. Conventionally, the safety and effectiveness of a particular treatment regimen for a particular patient is not known until after the treatment has been administered and its outcome evaluated, at which point it is too late to make any needed adjustments to enhance anti-tumor response and/or to enhance safety.

In various embodiments, the disclosed approach uses a surrogate for a therapeutic compound in predicting what dosage or other treatment protocol is expected to provide optimal (or otherwise desirable) results, in which the benefits (e.g., anti-tumor response or other desired effects) outweigh the costs (e.g., harm to healthy tissues or other undesirable effects). As further discussed below, a surrogate of a compound does not have the same benefits/toxicities as the compound (e.g., desirable therapeutic effects and undesirable side effects) in a patient, but instead exhibits pharmacological properties in patients that are sufficiently similar to the compound, thus serving as a useful proxy for how the patient would receive or otherwise react in a particular manner (e.g., uptake by targeted tissue) to the compound itself. For example, in some embodiments, a non-therapeutic radioisotope of a therapeutic compound can serve as a surrogate or proxy of the therapeutic compound (examples of which are provided below). The surrogate can be administered, and its pharmacological or other effects evaluated, to determine whether to administer the compound and, if so, to determine suitable dosage (e.g., number of times to be administered and amount to be administered each time), timing of administrations, and/or other treatment protocols for the compound.

Evaluating how a patient will react to a surrogate, however, can be a time consuming and undesirable process for the patient. The patient may comprise a genetic mutation that regulates uptake of the surrogate, such as BRAF V600E. In some embodiments, the patient has received or is receiving a MEK inhibitor and/or a BRAF inhibitor. Examples of BRAF inhibitors include, but are not limited to GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436). Examples of RAF/MEK/ERK inhibitors include, but are not limited to Vemurafenib (Zelboraf) and Dabrafenib (GSK2118436), Encorafenib, TAK-632, PLX4720, MLN2480, Cobimetinib (GDC-0973), MEK 162, R05126766, GDC-0623, VTX11e, Selumetinib (AZD6244), PD0325901, Trametinib (GSK1120212), U0126-EtOH, PD184352 (CI-1040), Refametinib, PD98059, BIX02189, Binimetinib, Pimasertib (AS-703026), SL327, BIX02188, AZD8330, TAK-733, PD318088, SCH772984, and FR 180204.

For example, if a radioisotope of a compound is administered as a surrogate of the compound, several sets of images (each set comprising one or more images captured during a separate visit at a different day and/or at a different time) may be needed to track the surrogate's progress in the patient. These can be inconvenient at best, and potentially harmful if the patient is exposed to x-rays or other radiation during the imaging process. In various embodiments, a biomarker may be generated for use in better evaluating how the patient responds to the surrogate using one set (or otherwise fewer sets) of one or more images. The biomarker may be generated by imaging subjects in a cohort of subjects. Each subject may have multiple lesions of a certain type, and may receive (or may have received) the compound or its surrogate. Images at multiple (e.g., 4 or more) time points following administration of the compound or its surrogate may be captured or otherwise obtained, and used to determine uptake of the compound or its surrogate by the lesions of each subject. The uptake of the compound or surrogate by the subjects at different time points could be modeled to provide a biomarker that could subsequently be used for dosage predictions for patients with one image (or multiple images) taken at only a single time point (or otherwise fewer time points than would otherwise be needed). In example embodiments, the model may involve an estimation equation (e.g., a generalized estimating equation (GEE)) model that is fitted on the log-transformed values of standardized uptake value (SUV) of a plurality of tumor lesions that have been contacted with a dose of radioisotope (e.g., I-124) and their corresponding log-transformed values of the variable area under curve (AUC) at a particular time period (e.g., 48 hours). Generation and use of a biomarker helps optimize or otherwise enhance patient care.

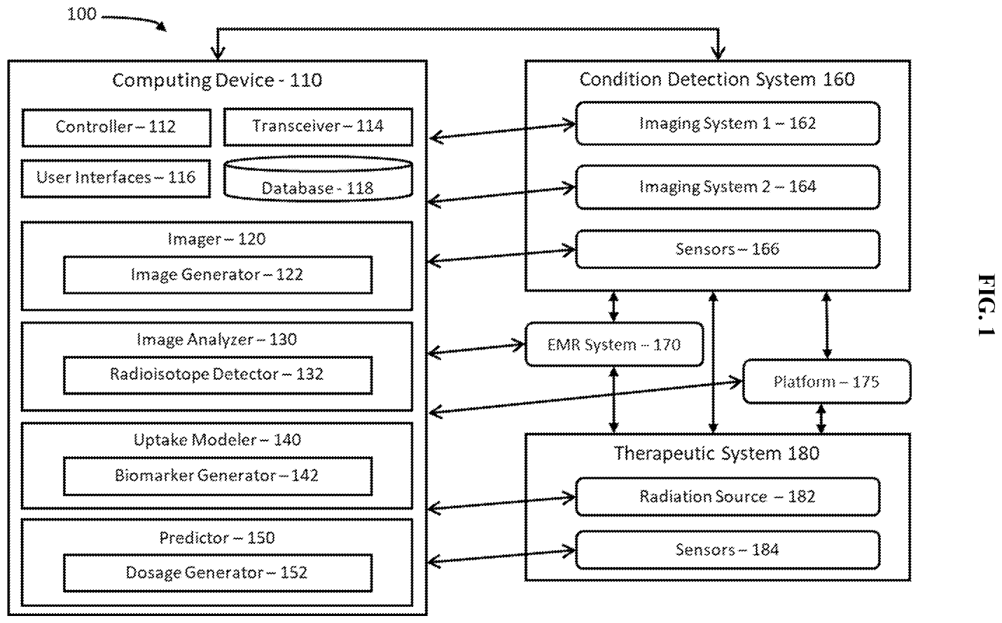

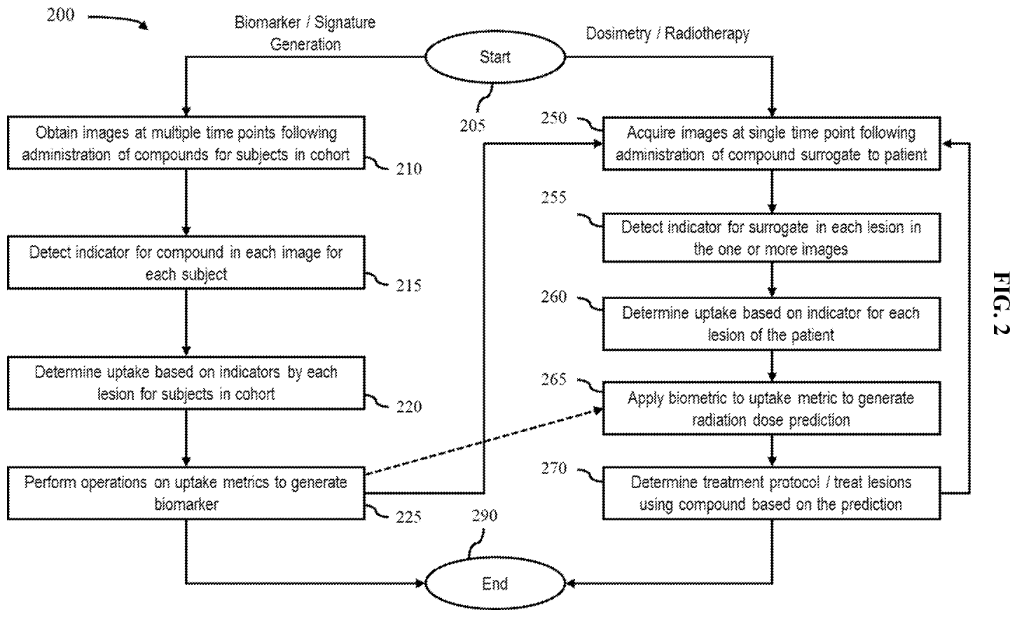

Referring to FIG. 1, in various embodiments, a system 100 may include a computing device 110 (or multiple computing devices, co-located or remote to each other), a condition detection system 160, an electronic medical record (EMR) system 170, a platform 175, and a therapeutic system 180. The computing device 110 (or multiple computing devices) may be used to control and/or exchange signals and/or data with condition detection system 160, EMR system 170, platform 175, and/or therapeutic system 180, directly or via another component of system 100. In certain embodiments, computing device 110 may be used to control and/or exchange data or other signals with condition detection system 160, EMR system 170, platform 175, and/or therapeutic system 180. The computing device 110 may include one or more processors and one or more volatile and non-volatile memories for storing computing code and data that are captured, acquired, recorded, and/or generated. The computing device 110 may include a controller 112 that is configured to exchange control signals with condition detection system 160, EMR system 170, platform 175, therapeutic system 180, and/or any components thereof, allowing the computing device 110 to be used to control, for example, capture of images, acquisition of signals by sensors, positioning or repositioning of subjects and patients, recording or obtaining subject or patient information, and applying therapies.

A transceiver 114 allows the computing device 110 to exchange readings, control commands, and/or other data, wirelessly or via wires, directly or via networking protocols, with condition detection system 160, EMR system 170, platform 175, and/or therapeutic system 180, or components thereof. One or more user interfaces 116 allow the computing device 110 to receive user inputs (e.g., via a keyboard, touchscreen, microphone, camera, etc.) and provide outputs (e.g., via a display screen, audio speakers, etc.). The computing device 110 may additionally include one or more databases 118 for storing, for example, signals acquired via one or more sensors, biomarker signatures, etc. In some implementations, database 118 (or portions thereof) may alternatively or additionally be part of another computing device that is co-located or remote and in communication with computing device 110, condition detection system 160, EMR system 170, platform 175, and/or therapeutic system 180 or components thereof.

Condition detection system 160 may include a first imaging system 162 (which may be or may include, e.g., a positron emission tomography (PET) scanner, a single photon emission computed tomography (SPECT) scanner, a magnetic resonance imaging (MRI) scanner, a computed tomography (CT) scanner, and/or other imaging devices and/or sensors), a second imaging system 164 (which may be or may include, e.g., a PET scanner, a SPECT scanner, an MRI scanner, a CT scanner, and/or other imaging devices and/or sensors), and sensors 166 (which may detect, e.g., a position or motion of a patient or other states or conditions). Therapeutic system 180 may include a radiation source for external beam therapy (e.g., orthovoltage x-ray machines, Cobalt-60 machines, linear accelerators, proton beam machines, neutron beam machines, etc.) and/or one or more other treatment devices. Sensors 184 may be used by therapeutic system 180 to evaluate and guide a treatment (e.g., by detecting level of emitted radiation, a condition or state of the patient, or other states or conditions). In various implementations, components of system 100 may be rearranged or integrated in other configurations. For example, computing device 110 (or components thereof) may be integrated with one or more of the condition detection system 160, therapeutic system 180, and/or components thereof. The condition detection system 160, therapeutic system 180, and/or components thereof may be directed to a platform 175 on which a patient or other subject can be situated (so as to image the subject, apply a treatment or therapy to the subject, and/or detect motion by the subject). In various embodiments, the platform 175 may be movable (e.g., using any combination of motors, magnets, etc.) to allow for positioning and repositioning of subjects (such as micro-adjustments to compensate for motion of a subject or patient).

The computing device 110 may include an imager 120 configured to direct image capture and obtain imaging data. Imager 120 may include an image generator 122 that may convert raw imaging data from condition detection system 160 into usable medical images or into another form to be analyzed. Computing device 110 may include an image analyzer 130 configured to identify features in images or imaging data or otherwise make use of images or imaging data. The image analyzer 130 may include a radioisotope detector 132 that locates and quantifies a radioactive compound (or a surrogate thereof) in a patient or other subject (e.g., in a lesion). Computing device 110 may include a uptake modeler 140 that is configured to analyze data corresponding to images of subjects in a cohort. Uptake modeler 140 may include a biomarker generator 142 that processes imaging data and data on timing that a radioactive compound or surrogate thereof was administered to a patient (which may be obtained via EMR system 170) to generate a biomarker that can subsequently be used in dosimetry for patients. Computing device 110 also includes a predictor 150 configured to make dose predictions based on imaging data of patients. The predictor 150 may include a dosage generator 152 that applies a biomarker (e.g., one output by biomarker generator 142 and stored in database 118) to patient images or patient imaging data to make a prediction with respect to what radiation dose would have a desired anti-tumor effect (e.g., shrink or eliminate a tumor) for the patient.

Referring to FIG. 2, an example process 200 is illustrated, according to various potential embodiments. Various elements of process 200 may be implemented by or via system 100 or components. Process 200 may begin (205) with biomarker/signature generation, which may be implemented by or via imager 120, image analyzer 130, and uptake modeler 140, if a biomarker is not already available (e.g., in database 118), or if additional biomarkers are to be generated or added to database 118. Alternatively, process 200 may begin with dosimetry/radiotherapy for a patient, which may be implemented by or via imager 120, image analyzer 130, and predictor 150, if a suitable biomarker/signature has already been generated or is otherwise already available. In various embodiments, process 200 may comprise both biometric generation (e.g., steps 210-225) followed by dosimetry (e.g., steps 250-270).

At 210, images at multiple time points (e.g., four or more time points) for each subject (e.g., while on platform 175) in a cohort of subjects may be acquired (e.g., via imaging systems 162 and/or 164) to capture or represent uptake (by a lesion) of a compound surrogate, or the compound itself, at different time points following administration of the compound or the surrogate. Step 210 acquires (e.g., by or via imager 120), for each subject in the cohort, one or more images at each of multiple time points (e.g., 3, 4, 5, or more time points) following administration of the compound or surrogate.

At 215, for each time point, the images of each subject are analyzed (e.g., by or via image analyzer 130) to identify an indicator, such as a signal that varies from signals from other tissue and that is indicative of the presence of the compound or its surrogate. Depending on the type of image, the signal that serves as an indicator can be a change in coloration, contrast, intensity, energy, etc. in the image (or in the raw or processed imaging data). At 220, each indicator can be converted or transformed to a metric that represents a degree of uptake (of the compound or its surrogate) by the lesion. At 225, a model can be applied to the uptake metrics to generate a biomarker or other signature (e.g., by or via uptake modeler 140). As further discussed below, the biomarker allows the system to determine, for a particular level of uptake by a lesion of a patient at a given amount of time after administration of the surrogate, what radiation dose of the compound would be expected to have a desired anti-tumor effect for the lesions of the patient (without unacceptable side effects). The biomarker may be stored (e.g., in database 118) for subsequent use. Process 200 may end (290), or proceed to step 250 for use in dosimetry for radiotherapy. (As represented by the dotted line from step 225 to step 265, the biometric may subsequently be used to predict what radiation dose would have a desired anti-tumor effect in patients.)

At 250, one or more images of the patient may be acquired (e.g., using a PET scanner, a SPECT scanner, and/or a CT scanner of imaging system 162 and/or imaging system 164 in response to control signals from computing device 110, by or via imager 120). These images may be taken following administration of a surrogate of a radioactive compound to the patient. The one or more images may captured at a single time point (e.g., during a single visit by the patient to a facility suitably equipped to image the patient). Advantageously, this eases the burden on the patient (by only requiring one imaging session) and allows for dosimetry in a more efficient and timely manner, thereby enhancing patient care. At 255, the one or more images may be analyzed (e.g., by or via image analyzer 130) to detect an indicator of uptake of the surrogate by lesions of the patient. At 260, an uptake metric (e.g., based on a standardized uptake value (SUV) that may be normalized by lean body mass (SUL)) may be determined (e.g., by or via image predictor 150), and at 265, the biometric may be applied to the uptake metric to generate a radiation dose prediction. At 270, the radiation dose prediction may be used to determine a treatment protocol (e.g., a number of applications of radiotherapy at particular dosages), and/or radiotherapy may be administered using the compound (e.g., by or via therapeutic system 180). Process 200 may end (290), or return to step 250 (e.g., after administering a course of radiotherapy) for subsequent imaging and radiation dose prediction based on a current condition of the patient's lesions as may be changed following administration of the prior course of radiotherapy.

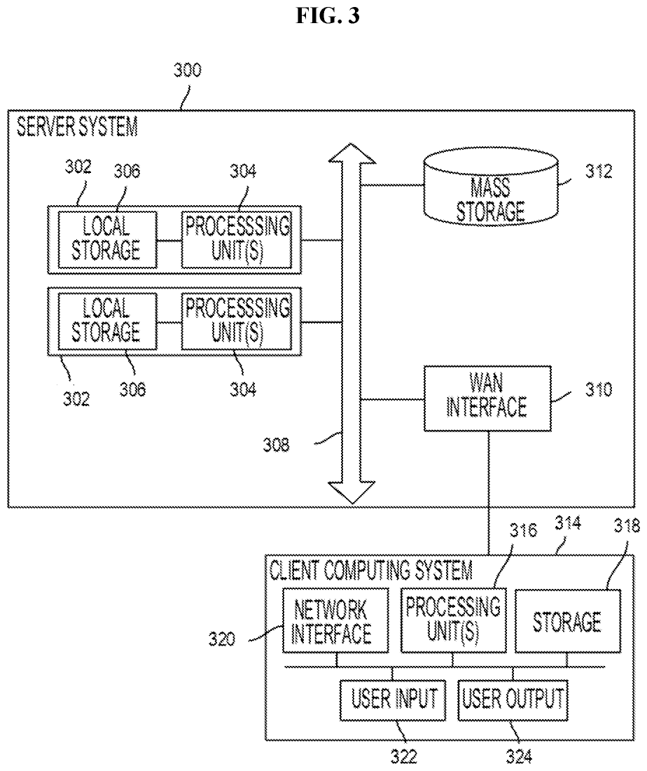

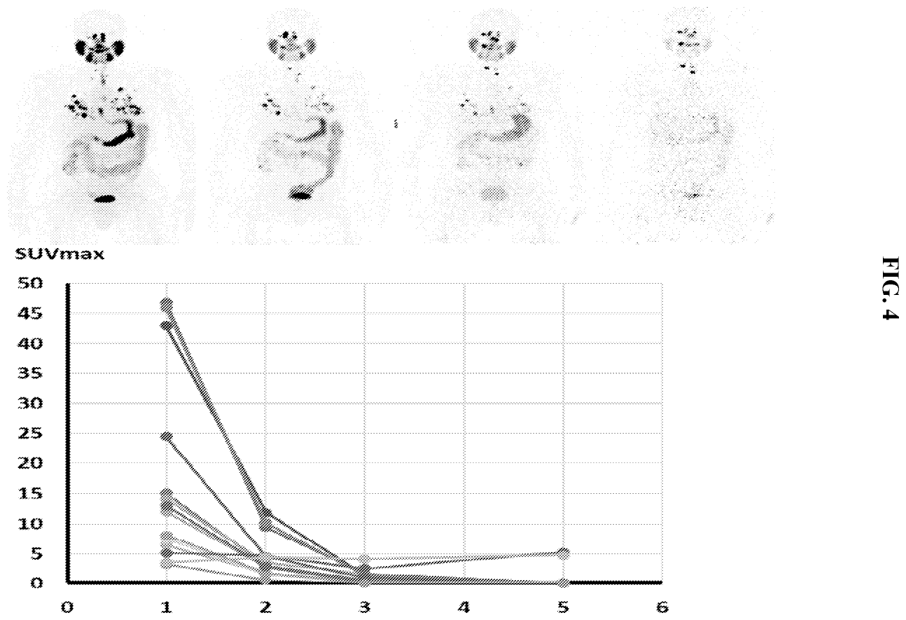

Various operations described herein can be implemented on computer systems, which can be of generally conventional design. FIG. 3 shows a simplified block diagram of a representative server system 300 (e.g., computing device 110) and client computer system 314 (e.g., computing device 110, condition detection system 160, imaging system 162, imaging system 164, sensors 166, EMR system 170, platform 175, therapeutic system 180, radiation source 182, and/or sensors 184) usable to implement various embodiments of the present disclosure. In various embodiments, server system 300 or similar systems can implement services or servers described herein or portions thereof. Client computer system 314 or similar systems can implement clients described herein.

Server system 300 can have a modular design that incorporates a number of modules 302 (e.g., blades in a blade server embodiment); while two modules 302 are shown, any number can be provided. Each module 302 can include processing unit(s) 304 and local storage 306.

Processing unit(s) 304 can include a single processor, which can have one or more cores, or multiple processors. In some embodiments, processing unit(s) 304 can include a general-purpose primary processor as well as one or more special-purpose co-processors such as graphics processors, digital signal processors, or the like. In some embodiments, some or all processing units 304 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 304 can execute instructions stored in local storage 306. Any type of processors in any combination can be included in processing unit(s) 304.

Local storage 306 can include volatile storage media (e.g., conventional DRAM, SRAM, SDRAM, or the like) and/or non-volatile storage media (e.g., magnetic or optical disk, flash memory, or the like). Storage media incorporated in local storage 306 can be fixed, removable or upgradeable as desired. Local storage 306 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 304 need at runtime. The ROM can store static data and instructions that are needed by processing unit(s) 304. The permanent storage device can be a non-volatile read-and-write memory device that can store instructions and data even when module 302 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 306 can store one or more software programs to be executed by processing unit(s) 304, such as an operating system and/or programs implementing various server functions or any system or device described herein.

“Software” refers generally to sequences of instructions that, when executed by processing unit(s) 304 cause server system 300 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 304. Software can be implemented as a single program or a collection of separate programs or program modules that interact as desired. From local storage 306 (or non-local storage described below), processing unit(s) 304 can retrieve program instructions to execute and data to process in order to execute various operations described above.

In some server systems 300, multiple modules 302 can be interconnected via a bus or other interconnect 308, forming a local area network that supports communication between modules 302 and other components of server system 300. Interconnect 308 can be implemented using various technologies including server racks, hubs, routers, etc.

A wide area network (WAN) interface 310 can provide data communication capability between the local area network (interconnect 308) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 306 is intended to provide working memory for processing unit(s) 304, providing fast access to programs and/or data to be processed while reducing traffic on interconnect 308. Storage for larger quantities of data can be provided on the local area network by one or more mass storage subsystems 312 that can be connected to interconnect 308. Mass storage subsystem 312 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 312. In some embodiments, additional data storage resources may be accessible via WAN interface 310 (potentially with increased latency).

Server system 300 can operate in response to requests received via WAN interface 310. For example, one of modules 302 can implement a supervisory function and assign discrete tasks to other modules 302 in response to received requests. Conventional work allocation techniques can be used. As requests are processed, results can be returned to the requester via WAN interface 310. Such operation can generally be automated. Further, in some embodiments, WAN interface 310 can connect multiple server systems 300 to each other, providing scalable systems capable of managing high volumes of activity. Conventional or other techniques for managing server systems and server farms (collections of server systems that cooperate) can be used, including dynamic resource allocation and reallocation.

Server system 300 can interact with various user-owned or user-operated devices via a wide-area network such as the Internet. An example of a user-operated device is shown in FIG. 3 as client computing system 314. Client computing system 314 can be implemented, for example, as a consumer device such as a smartphone, other mobile phone, tablet computer, wearable computing device (e.g., smart watch, eyeglasses), desktop computer, laptop computer, and so on.
example, client computing system 314 can communicate via WAN interface 310. Client computing system 314 can include conventional computer components such as processing unit(s) 316, storage device 318, network interface 320, user input device 322, and user output device 324. Client computing system 314 can be a computing device implemented in a variety of form factors, such as a desktop computer, laptop computer, tablet computer, smartphone, other mobile computing device, wearable computing device, or the like.

Processor 316 and storage device 318 can be similar to processing unit(s) 304 and local storage 306 described above. Suitable devices can be selected based on the demands to be placed on client computing system 314; for example, client computing system 314 can be implemented as a “thin” client with limited processing capability or as a high-powered computing device. Client computing system 314 can be provisioned with program code executable by processing unit(s) 316 to enable various interactions with server system 300 of a message management service such as accessing messages, performing actions on messages, and other interactions described above. Some client computing systems 314 can also interact with a messaging service independently of the message management service.

Network interface 320 can provide a connection to a wide area network (e.g., the Internet) to which WAN interface 310 of server system 300 is also connected. In various embodiments, network interface 320 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 322 can include any device (or devices) via which a user can provide signals to client computing system 314; client computing system 314 can interpret the signals as indicative of particular user requests or information. In various embodiments, user input device 322 can include any or all of a keyboard, touch pad, touch screen, mouse or other pointing device, scroll wheel, click wheel, dial, button, switch, keypad, microphone, and so on.

User output device 324 can include any device via which client computing system 314 can provide information to a user. For example, user output device 324 can include a display to display images generated by or delivered to client computing system 314. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 324 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 304 and 316 can provide various functionality for server system 300 and client computing system 314, including any of the functionality described herein as being performed by a server or client, or other functionality associated with message management services.

It will be appreciated that server system 300 and client computing system 314 are illustrative and that variations and modifications are possible. Computer systems used in connection with embodiments of the present disclosure can have other capabilities not specifically described here. Further, while server system 300 and client computing system 314 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. For instance, although specific examples of rules (including triggering conditions and/or resulting actions) and processes for generating suggested rules are described, other rules and processes can be implemented. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

### EXAMPLES

The present technology is further illustrated by the following Examples, which should not be construed as limiting in any way.

### Example 1: Experimental Methods

Population. The present study includes consecutive 21 patients studied under two different RB-approved protocols who underwent imaging. Patients who were considered candidates for RAI treatment of DTC were enrolled. All patients had histologically confirmed, metastatic thyroid cancer. See Table 1.

TABLE 1
Patient demographics

| Characteristic | N = 21¹ | |
|---|---|---|
| Age (years) | 57 | [22-85] |
| Sex | | |
| Female | 8 | (38%) |
| Male | 13 | (62%) |
| Stage | | |
| Stage IV | 20 | (95%) |
| T1N1M0 | 1 | (5%) |
| Thyroglobulin | 153 | [0-139,750] |
| Lesions | 11.0 | [3.0-23.0] |
| Maximum Tolerated Activity (mCi) | 426 | [47-896] |
| Treated | 15 | (71%) |
| ¹³¹I dose given (mCi) in treated patients | 194 | [46-407] |

¹Median [Range]; n (%)

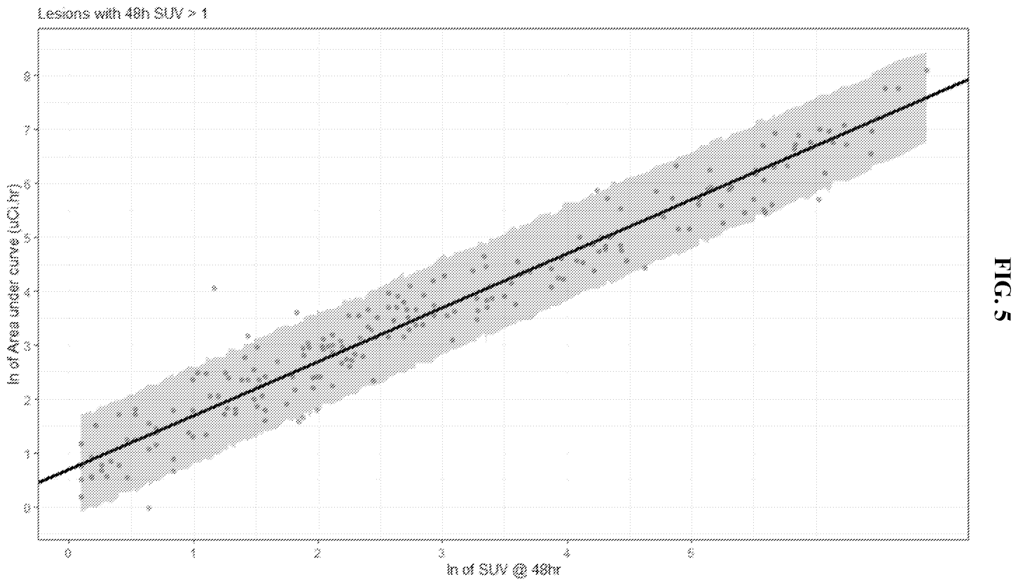

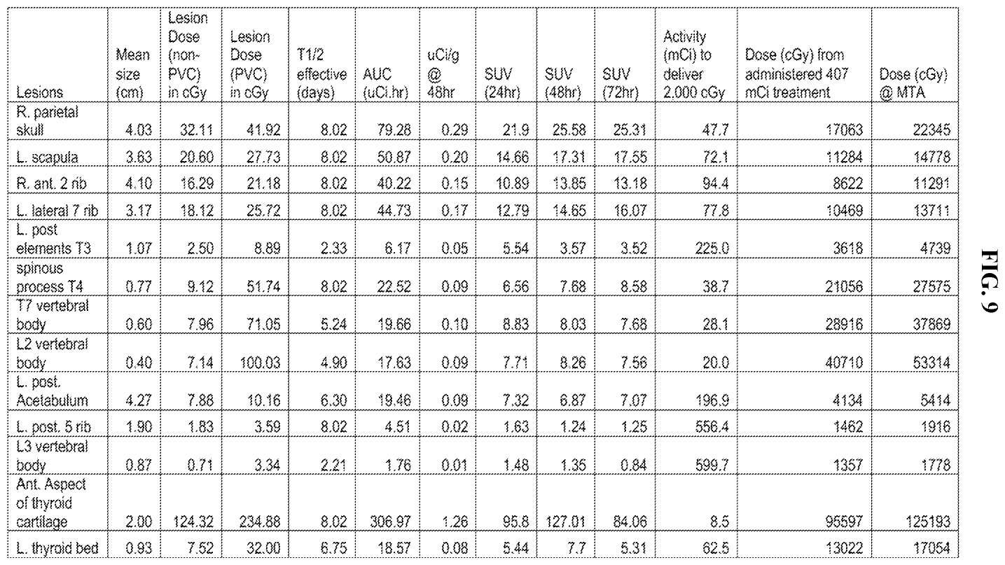

Dosimetry and estimation of average lesion uptake. A workflow was established for four-time point measurement of individual lesion radioiodine kinetics to generate area under the curve (AUC) estimation for accurate determination of the radiation dose in Gray (Gy) to individual lesions in patients undergoing RAI therapy for DTC. Positron emission tomography (PET/CT) imaging was performed after oral administration of 222 MBq (6.0 mCi) of ¹²⁴I—NaI (3D Imaging, Waco, TX). Whole-body imaging was performed at 4, 24, 48, and 96 hours after oral administration of the radioactivity (FIG. 4). Regions of interest were obtained over all visible lesions >0.5 cm within the body and multiple parameters were obtained for later analysis. The following parameters were captured in a database for each patient and each lesion: size in three dimensions (cm); standardized uptake value (SUV), maximum and average; lean body weight; and activity concentration in microcurie/gram. (FIG. 9). For these patients, the best clearance fitting curve was estimated using a dual exponential equation model comprising lesion uptake and clearance. Prior to integration, clearance fitting was adjusted to replace the decay constant of ¹²⁴I with ¹³¹I, the therapy isotope of interest, and then integrated to obtain the AUC. This AUC was multiplied by the equilibrium dose constant for the non-penetrating P-emissions of ¹³¹I, yielding the lesion-absorbed dose in Gy.

Predicting AUC based on a single time point. One goal of this study was to use a single time point to predict the overall area under the radioiodine lesion activity retention curve to estimate the radiation-absorbed dose to lesions without the need for costly additional PET/CT imaging to fully characterize the kinetic behavior. First, the approach starts with estimating the linear relationship between the dosimetry as summarized by the AUC based on four measured time points and the activity measured at one time point, called the predictor (e.g., SUV at 48 h; see FIG. 5). For this estimation, the unit is the lesion, and a generalized estimating equation approach is used to estimate the parameters (intercept, slope, and robust variance matrix) accounting for the correlation between lesions in the same patient. Log-transformed values of the uptake and doses are used to ensure the data are normally distributed.

The linear model is as follows, where the errors ε_ij_ are correlated, y_ij_ is the logarithm of the AUC value, and x_ij_ is the uptake measured at one time-point, e.g., the logarithm of 48 h SUV measured for the lesion j from patient i:

$$y\_ij \_ = {x}^{′} \_ij \_β + ε\_ij \_$$

Second, using the estimations for R and the covariance matrix, a prediction interval (PI) is calculated. A PI differs from a confidence interval, as it aims to predict with 95% confidence where future measurements will fall. In our case, if we observed the same value of SUV at 48 h for 100 new lesions, the PI is the range in which 95 of those lesions' AUCs will be found. As difficulties arose when analytically constructing the PI, simulated prediction was used to calculate PIs following the steps detailed in Gelman and Hill, Data Analysis Using Regression and Multilevel/Hierarchical Models (1st Edition, 2007) and summarized in Appendix A.

To validate the accuracy of the prediction, a leave-one-patient-out cross-validation approach was used. For each patient i (i=1, . . . , n), the linear regression parameters are re-estimated using n−1 patients (excluding i), and PIs are calculated for each lesion in patient i based on their measured predictor values.

For those lesions, the actual observed AUC is then compared to the PI. When using 95% PI, it is expected that 95% of the observed values will fall into the corresponding PIs; i.e., 5% will be outside the prediction. In addition, for each left-out patient, an error of prediction is calculated corresponding to the squared difference between the predicted and true AUC values for each lesion. This squared error is average over all the lesions of all the patients to obtain a cross-validated error.

Range of ¹³¹I activity to treat a chosen efficacy rate for RAI in mDTC patients. A multi-disciplinary team of nuclear medicine specialists, endocrinologists, medical oncologists, and medical physicists met weekly to make treatment decisions based on the PET data generated, including data from the methods disclosed herein.

The target radiation dose of 2000 cGy was chosen because doses above this level are often used as the threshold for patients to receive further radioiodine treatment. Based on the PI now available for the AUC, a simple calculation of the relationship between ¹²⁴I and ¹³¹I uptake can yield a PI for the dose [d_{low}−d_{high}]. For a 95% PI, the interval shows the ¹³¹I activity that will ensure a dose of 2000 cGy in 95% of the lesions with the corresponding measured uptake. Thus, the higher boundary (d_{high}) corresponds to the activity to treat 97.5% of the lesions with the given uptake. By varying this boundary, it is possible to select an activity that will target 95%, 90%, or fewer of the lesions. This provides the treating physician with information necessary to select a balance between the activity needed and the predicted efficacy.

Maximum Tolerated Activity (MTA). For over 60 years, MSK clinicians have employed a series of simple dosimetry benchmarks that provided guidelines for MTA. These guidelines have shown a remarkable safety record with respect to avoidance of serious toxicity to lung and bone marrow during high-dose RAI treatment for differentiated thyroid cancer. The benchmarks are described in Table 2.

TABLE 2
Maximum Tolerated Activity

Dosimetry measurements based on total and blood clearances 200 rads to
blood
Body retention less 120 mCi in 48 hrs. 80 mCi in diffuse lung diseases
No limit of single dose
Usually under 0.5 Ci
Less than 300 mCi if salivary complication is a concern
*Benua, Sonenberg, Rawson et al: American J Roentgenology Radium Therapy and Nuclear Medicine 1962; 87 (1): 171-182

To perform these studies, serial blood samples and total body measurements were conducted to determine β- and photon radiation dose contributions to blood (a proxy for the dose-limiting bone marrow) from a pre-therapy tracer administration of either ¹²⁴I or ¹³¹I. This MTA information provides the prescribing physician with an upper bound for the administered treatment activity of ¹³¹I, which can be used in combination with statistical lesion dose predictions to select the most appropriate treatment activity for that specific patient.

### Example 2: Efficacy of the Lesional Dosimetry Methods of the Present Technology

Patients. At present, data from 208 lesions in 21 individual patients was analyzed. The median age was 57 years (range: 22-85), and 62% were male. All but one had stage IV disease. Patients had between 3 and 23 lesions (median=11). Overall, 71% (15 patients) were treated by ¹³¹I, with doses ranging from 46 to 407 mCi.

The results of the full lesion dosimetric analysis based on the four ¹²⁴I PET imaging time points were determined. An example dataset of most of the parameters determined for patient #1 is shown in FIG. 9. The full parameter data set consists of: anatomical descriptor, mean size (cm), lesion dose per projected unit millicurie of administered ¹³¹I activity with and without partial volume correction (5), half-life based on a linear slop between day 3 and 5 as well as based on exponential curve fitting, area under the curve based on an integrated curve fit, estimated activity per gram at 48 and 72 hours post-administration, SUV and SUL (based on lean body mass) at 24, 48, and 72 hours post-administration, the administered activity to deliver 2,000 cGy, the radiation dose estimate resulting from the actual therapeutic amount administered to the patient, and the maximum projected dose that could have been achieved had the maximum tolerated activity been administered. This dataset was used as the input to derive a statistical model to predict the radiation dose to lesions. The dosimetry summary for all patients is given in Table 3.

TABLE 3
Patient Dosimetry Summary

| | | | | | | | | | | Admin | | |
|---|---|---|---|---|---|---|---|---|---|---|---|---|
| | Lesion | | | | | | | Activity to give | | Activity | | |
| Patient | # | Size (cm) | | Teff (d) | | SUV(48 h) | | 20 Gy (mCi) | | (mCi) | Dose (Gy) | |
| 1 | 13 | 1.3 | (0.4-4.27) | 8.02 | (2.21-8.02) | 8.03 | (1.24-127) | 72.1 | (8.51-599.7) | 407 | 112 | (13.6-955) |
| 2 | 18 | 0.52 | (0.22-2.45) | 4.28 | (0.78-8.02) | 80.1 | (0.7-983) | 3.0 | (0.43-1132) | 194 | 1,285 | (3.4-9,062) |
| 3 | 14 | 0.87 | (0.63-1.97) | 0.45 | (0.4-7.42) | 3.25 | (0.6-11.9) | 135 | (30.2-351) | Not Treated | N/A | |
| 4 | 12 | 0.68 | (0.30-1.67) | 3.16 | (1.31-7.88) | 7.4 | (3.6-26.5) | 61.3 | (18.95-165.3) | 222.7 | 73 | (26.9-406) |
| 5 | 10 | 0.72 | (0.30-2.47) | 1.48 | (1.01-3.57) | 1.85 | (0.50-26.4) | 126 | (12.7-1451) | Not Treated | N/A | |
| 6 | 16 | 0.95 | (0.60-4.37) | 1.59 | (0.43-4.38) | 27.8 | (0.50-119) | 26.38 | (2.84-2959) | 196.8 | 151 | (1.8-1387) |
| 7 | 23 | 0.97 | (0.30-1.97) | 2.48 | (0.99-7.89) | 35 | (10-783) | 20.4 | (1.06-1854) | 236.1 | 232 | (18.5-122631) |
| 8 | 6 | 0.67 | (0.23-1.1) | 4.41 | (1.74-6.29) | 1.73 | (0-36.6) | ND | (74.1-infinity) | 138.2 | 18.6 | (0-273) |
| 9 | 8 | 0.58 | (0.43-1.27) | 3.03 | (1.1-7.14) | 4.35 | (1.6-11.5) | 99.3 | (61.7-435) | Not Treated | N/A | |
| 10 | 4 | 2.37 | (1.7-4.13) | 1.26 | (0.82-2.29) | 26.2 | (0.70-515) | 9.87 | (0.034-24.96) | 158.4 | 49.2 | (1.27-930) |
| 11 | 11 | 0.40 | (0.30-2.8) | 2.98 | (1.47-4.06) | 11.30 | (6.9-189) | 15.37 | (4.11-59.2) | 171.4 | 223 | (57.8-834) |
| 12 | 3 | 0.87 | (0.3-1.3) | 3.8 | (0.52-7.89) | 0.7 | (0.50-5.3) | 179.8 | (71.8-745) | Not Treated | N/A | |
| 13 | 13 | 0.90 | (0.43-3.43) | 1.55 | (0.86-5.02) | 1.30 | (0.50-4.2) | 1175 | (46.9-1695) | Not Treated | N/A | |
| 14 | 5 | 0.30 | (0.23-1.13) | 1.74 | (0.54-1.85) | 0.90 | (0.30-3.20) | 395 | (69.1-2026) | Not Treated | N/A | |
| 15 | 17 | 1.57 | (0.77-2.37) | 3.79 | (1.87-5.02) | 385.3 | (13.5-707) | 2.47 | (1.24-27.59) | 46.08 | 372 | (33.4-744) |
| 16 | 6 | 1.22 | (0.60-3.83) | 4.77 | (2.29-8.02) | 8.30 | (2.6-20.3) | 99.7 | (50.7-148.9) | 220.8 | 44.3 | (31.3-87.1) |
| 17 | 6 | 0.58 | (0.27-0.77) | 8.04 | (1.22-8.02) | 0.80 | (0-4.80) | ND | (91.8-infinity) | 392.6 | 21.15 | (0-85.5) |
| 18 | 12 | 0.35 | (0.27-0.97) | 1.69 | (1.22-2.86) | 25.1 | (3.1-78.0) | 10.87 | (2.27-45.3) | 147.9 | 272 | (65.4-1306) |
| 19 | 17 | 0.83 | (0.40-2.0) | 1.55 | (1.09-6.43) | 204.6 | (76.5-629) | 3.70 | (0.93-10.27) | 98 | 529 | (192-2086) |
| 20 | 13 | 0.50 | (0.20-1.97) | 1.74 | (1.22-2.81) | 2.30 | (0.50-15.3) | 65.1 | (21.9-1935) | 239 | 73.4 | (2.47-218.4) |
| 21 | 10 | 0.68 | (0.43-1.50) | 4.63 | (2.46-8.02) | 10.8 | (1.1-21.7) | 77.3 | (14.75-295) | 398.4 | 104.1 | (26.99-540) |

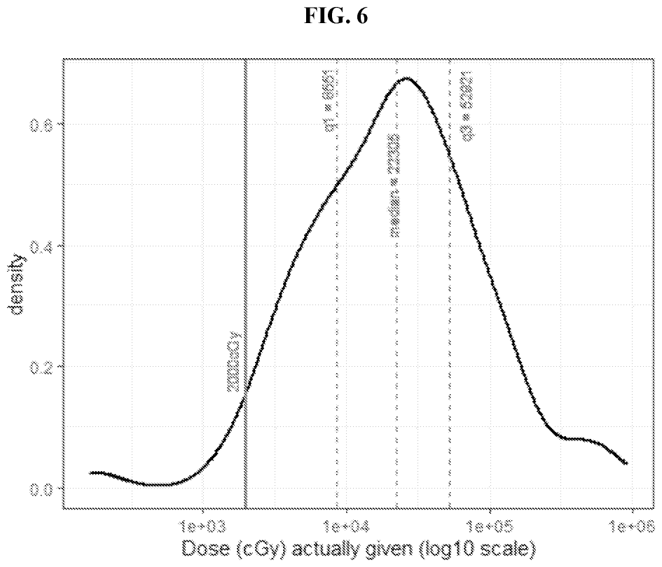

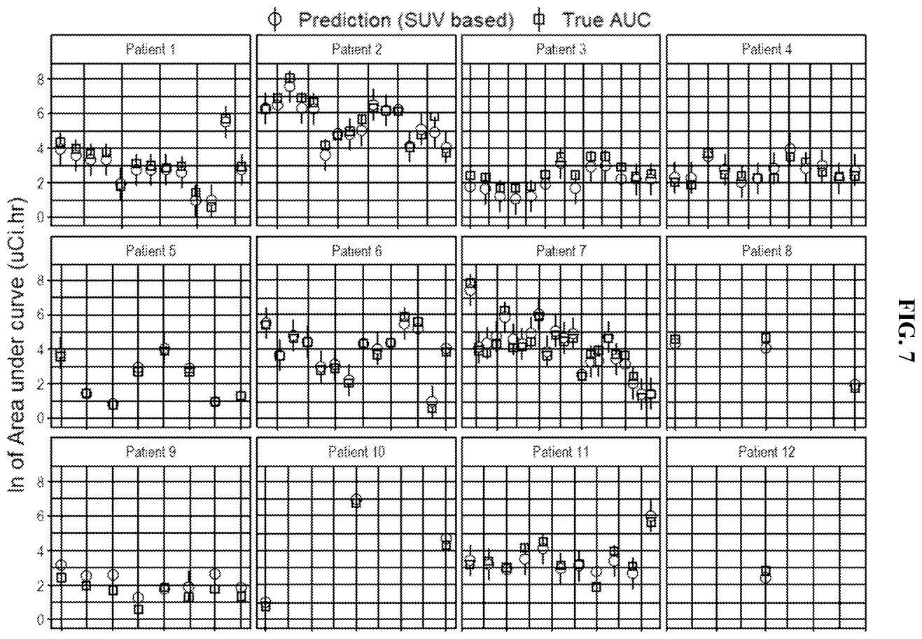

Overall, 15 patients were treated following the dosimetry. The median lesion dose was 22,305 cGy (interquartile range: 8551-52,921, range: 163-906,218 cGy; FIG. 6). Assuming a linear relationship between the lesion uptake profiles measured by ¹²⁴I PET dosimetry and the ¹³¹I therapy administered to the patients, it was determined that of the 169 treated lesions, 163 (96%) received a dose greater than 2000 cGy.

Prediction of activity to deliver 2000 cGy. An operational definition in the methods disclosed herein is that a patient with any lesion with a predicted dose of >2000 cGy would likely respond to treatment. The results described herein demonstrated that patients selected in this way had a high probability of durable partial RECIST response (75%) or complete reduction (100%). A total of 208 lesions are included in the analysis. The prediction is limited for lesions with an SUV >1, as no treatment is planned for lesions with no uptake. Lesions that received no treatment (n=39) were included in the analysis, as prediction is still feasible. For the dataset analyzed, the estimated regression coefficient (slope) is 1.002 (robust se=0.024; 95% confidence interval: 0.954 to 1.049; p<0.0001). The full predicted value of AUC based on 48 h uptake can be calculated as: AUC__=exp__0.697+1.002. In (SUV_48_)___.

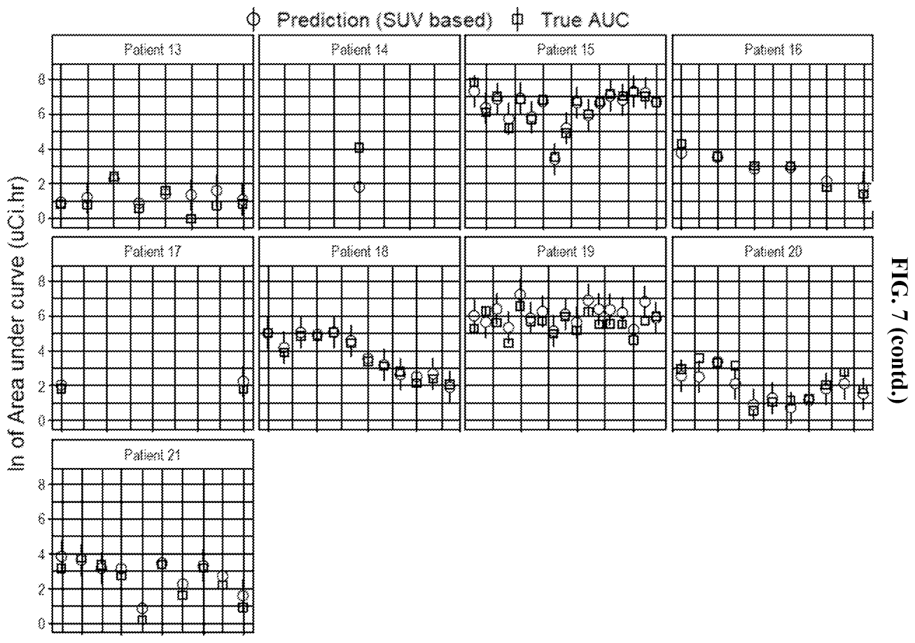

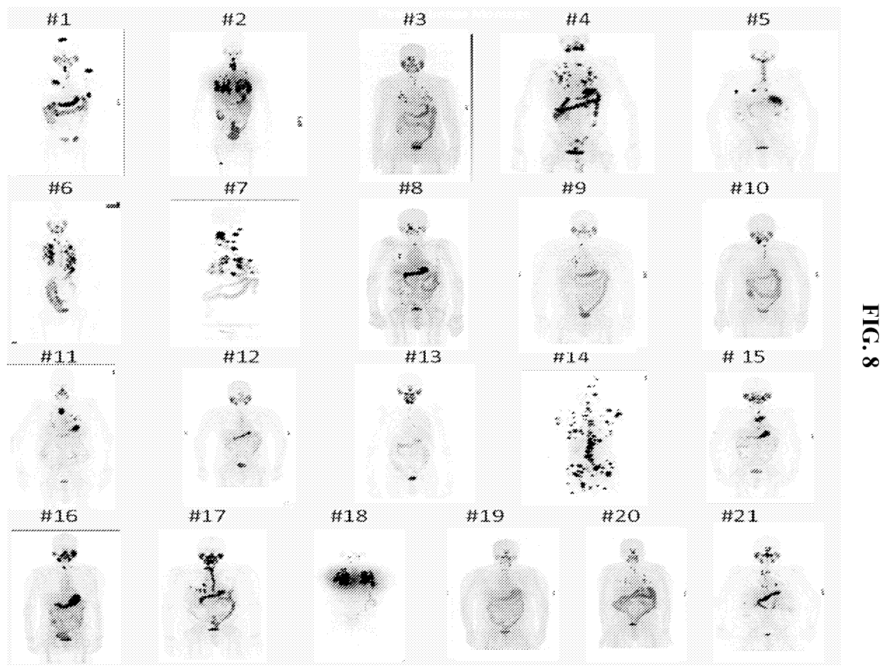

FIG. 5 illustrates each lesion according to its ln SUV_48_value as measured, and the In-AUC as measured based on the four time points. Each dot marks the lesions from a patient. The black line is the average regression line according to the GEE estimate, and the gray area represents the 95% PI. As expected, a few dots fall outside the PI, but the PI covers the majority of the lesions. To assess whether this prediction is accurate for new patients, the LOO cross-validation was done for all 21 patients (FIG. 7). For each patient (separate quadrant), the AUC as predicted by the model disclosed herein is represented by a circle and its accompanying line represents the 95% PI. The squares represent the actual AUC as measured on the lesion. In all but 12 of the 208 lesions (from 7/21 patients), the actual AUC based on four time points fell into the 95% PI based on the 48 h time point, showing good performance of our model and the feasibility of using one time point to guide treatment decisions. The estimated radiation-absorbed dose achieved at the mCis actually administered were consistent in that 96% of lesions were treated with RAI to >2000 cGy, which was the prescribed treatment cGy dose. Table 4 shows the predicted required activities for a plausible range of lesion uptakes.

TABLE 4
Prediction of AUC based on the 48 h SUV measured, and corresponding
activity to be administered to deliver 20 Gy

| | AUC (uCi · hr/g per mCi) Activity (mCi) to deliver 20 Gy | | | | | |
|---|---|---|---|---|---|---|
| | | | | 95% PI | | |
| SUV at | Mean | 95% | Mean | (97.5% upper | 95% upper | 90% upper |
| 48 h | Estimate | PI | Estimate | bound) | bound | bound |
| 2 | 4.0 | 1.5-9.1 | 1228.1 | 542.4-3200.4 | 2612.8 | 2236.5 |
| 4 | 8.1 | 3.3-20.3 | 613.2 | 243.5-1474.8 | 1278.0 | 1066.4 |
| 6 | 12.1 | 4.8-29.2 | 408.5 | 168.9-1032.0 | 890.6 | 759.4 |
| 8 | 16.1 | 6.4-38.8 | 306.2 | 127.4-775.6 | 692.4 | 570.5 |
| 10 | 20.2 | 8.6-48.4 | 244.8 | 102.1-576.6 | 517.0 | 431.4 |
| 15 | 30.3 | 11.9-71.0 | 163.1 | 69.6-416.2 | 355.5 | 292.3 |
| 20 | 40.4 | 16.1-97.1 | 122.2 | 50.9-306.9 | 254.8 | 217.8 |
| 30 | 60.6 | 24.3-147.4 | 81.4 | 33.5-202.9 | 179.1 | 153.1 |
| 50 | 101.2 | 40.8-252.9 | 48.8 | 19.5-121.1 | 102.7 | 87.9 |
| 100 | 202.6 | 82.3-479.6 | 24.4 | 10.3-60.0 | 53.1 | 45.3 |
| 200 | 405.8 | 164.2-951.2 | 12.2 | 5.2-30.1 | 26.0 | 21.5 |
| 300 | 609.2 | 257.8-1476.2 | 8.1 | 3.3-19.2 | 16.7 | 14.5 |

SUVs at 24 h and 72 h were also assessed; however, they showed a higher prediction error than the 48 h time point (cross-validated squared error of 0.472 and 0.327 for 24 h and 72 h, respectively, versus 0.223 for 48 h; Table 5).

TABLE 5
Estimate of linear regression parameters, prediction error, and cross-
validated prediction error, and estimated required activity to deliver
2000 cGy for different predictors using one time point

| | | | | | CV |
|---|---|---|---|---|---|
| | | | Robust | Squared | Squared |
| Timepoint | N | Slope | se | Error | Error |
| uCi 24 h | 231 | 1.018 | 0.050 | 0.615 | 0.665 |
| uCi 48 h | 231 | 0.934 | 0.043 | 0.443 | 0.484 |
| uCi 72 h | 231 | 0.859 | 0.051 | 0.679 | 0.761 |
| SUV 24 h | 217 | 1.057 | 0.045 | 0.436 | 0.472 |
| SUV 48 h | 208 | 1.002 | 0.024 | 0.204 | 0.223 |
| SUV 72 h | 193 | 0.963 | 0.039 | 0.292 | 0.327 |
| SUL 24 h | 211 | 1.062 | 0.046 | 0.403 | 0.434 |
| SUL 48 h | 200 | 1.013 | 0.028 | 0.207 | 0.225 |
| SUL 72 h | 186 | 0.955 | 0.044 | 0.301 | 0.338 |

uCi: micro-Curie; h: hour; se = standard-error; CV: cross-validated; cGy: centi-Gray.

Using the activity measured in μCi led to higher prediction error, while the use of SUL led to very similar prediction errors to those of SUV (Table 5).

The addition of a second, later time point to account for the clearance of each patient did not seem to improve the prediction error (Table 6).

TABLE 6
Estimate of the prediction error and cross-validated prediction
error, and estimated required activity to deliver 2000 cGy
for different predictors that using time points

| | | | CV |
|---|---|---|---|
| | | Squared | Squared |
| Timepoint | N | Error | Error |
| uCi 48 h and uCi 72 h | 231 | 0.449 | 0.514 |
| uCi 48 h and ratio 72/48 | 231 | 0.449 | 0.515 |
| SUV 48 h and SUV 72 h | 208 | 0.226 | 0.298 |
| SUV 48 h and ratio 72/48 | 208 | 0.226 | 0.299 |
| SUL 48 h and SUL 72 h | 200 | 0.224 | 0.296 |
| SUL 48 h and ratio 72/48 | 200 | 0.224 | 0.297 |

uCI: micro-Curie; h: hour; CV: cross-validated; cGy: centi-Gray.

The better predictor was the SUL measured at 48 h and 72 h, with a cross-validated squared error of 0.296.

The methods of the present technology are applicable to normal organ dose i.e., Bone Marrow; salivary glands, and other targeted radiotherapeutic (TRT) agents for both individual lesion dose and organ dose. e.g. Lutathera (NET); PSMA-617 (Prostate); DOTA and SADA PRIT.

Taken together, the methods disclosed herein predict the average uptake value of a surrogate (e.g., ¹²⁴I) as measured by the AUC (in microcurie-hour per gram/mCi or SUV) that is drawn based on four timepoints (24, 48, 72, and 96 hours) using a unique timepoint (e.g., 48 hours). Further, based on the predicted uptake, one can determine the prescribed administered activity required (in mCi, or MBq of ¹³¹I) to reach a desired radiation-absorbed dose in cGy (e.g., >2000 cGy) to treat the patient's lesion(s).

Accordingly, the lesional dosimetry methods of the present technology accurately predict the amount of radioactivity to be administered (in MBq) to achieve a specific radiation-absorbed dose (cGy) to individual lesions.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A computer-implemented dosimetry method comprising:
detecting, in a single-time-point medical image of a patient, for each cancerous lesion of the patient, an indicator of a surrogate of a radiotherapeutic compound, the medical image captured within a predetermined time range following administration of the surrogate to the patient;
determining, based on each indicator, an uptake metric corresponding to uptake of the surrogate by each corresponding cancerous lesion;
generating, based on the predetermined time range and the uptake metric, using a dosimetry biomarker based on uptake of the surrogate by subjects in a cohort of subjects, a radiation dose prediction for administration of the radiotherapeutic compound to the patient; and
providing the radiation dose prediction for subsequent use by a healthcare professional in determining a treatment protocol for treating the cancerous lesions of the patient using the radiotherapeutic compound, wherein the surrogate is an isotope of the radiotherapeutic compound, optionally wherein the surrogate is Iodine-124, and the radiotherapeutic compound is Iodine-131.

2. The computer-implemented method of claim 1, wherein the uptake metric is based on a standardized uptake value (SUV), optionally wherein the uptake metric is the SUV normalized by lean body mass (SUL).

3. (canceled)

4. The computer-implemented method of claim 1, wherein the medical image is captured using a medical imaging system comprising at least one of a positron emission tomography (PET) scanner or a single photon emission computed tomography (SPECT) scanner, and wherein the medical image comprises at least one of a PET scan or a SPECT scan or
wherein the biomarker indicates dosages corresponding to different uptake metrics; or
wherein the radiotherapeutic compound is I¹³¹, PSMA-617, lutetium Lu¹⁷⁷ m dotatate, or radiolabeled DOTA hapten.

5. (canceled)

6. (canceled)

7. (canceled)

8. The computer-implemented method of claim 1, further comprising generating the biomarker by:
for each subject in the cohort of subjects, obtaining a plurality of images captured at a plurality of times following administration of the surrogate, each subject having one or more cancerous lesions;
detecting, in the plurality of images, for each cancerous lesion of each subject, a surrogate indicator corresponding to presence of the surrogate in each cancerous lesion;
determining, based on the surrogate indicators for the subjects in the cohort of subjects, one or more surrogate uptake metrics indicative of uptake of the surrogate by the corresponding cancerous lesions of the subjects; and
performing operations on the uptake metrics to generate the biomarker for predicting radiation dose for the radiotherapeutic compound, the operations comprising application of a generalized estimating equation (GEE) model to the uptake metrics.

9. The computer-implemented method of claim 8, wherein the plurality of times comprises four times following administration of the surrogate; or wherein the operations further comprise generating areas under the curve (AUCs) based on the surrogate uptake metrics.

10. (canceled)

11. (canceled)

12. A computer-implemented method comprising:
for each subject in a cohort of subjects, obtaining a plurality of images captured using a medical imaging system at a plurality of times following administration of at least one of (i) a radiotherapeutic compound, or (ii) a surrogate for the radiotherapeutic compound, each subject having one or more cancerous lesions;
detecting, in the plurality of images, for each cancerous lesion of each subject, a surrogate indicator corresponding to presence of the surrogate in each cancerous lesion;
determining, based on the surrogate indicators, one or more surrogate uptake metrics indicative of uptake of the surrogate by the corresponding cancerous lesions;
performing operations on the uptake metrics to generate a biomarker for predicting radiation dose for the radiotherapeutic compound, the operations comprising application of an estimation model to the uptake metrics; and
providing the biomarker for subsequent prediction of radiation dose in determining treatment protocols for patients with one or more cancerous lesions based on uptake metric and amount of time following administration of the surrogate to the patients.

13. The method of claim 12, wherein the plurality of images are captured following administration of only the surrogate of the radiotherapeutic compound to each subject and not the radiotherapeutic compound; or
wherein the estimation model is based on a generalized estimating equation (GEE); or
wherein the plurality of times comprises four times following administration of the surrogate; or
wherein the operations further comprise generating areas under the curve (AUCs) based on the surrogate uptake metrics; or
wherein the medical imaging system comprises a positron emission tomography (PET) scanner or a single photon emission computed tomography (SPECT) scanner, and the medical image comprises a PET scan or a SPECT scan; or
wherein the surrogate is an isotope of the radiotherapeutic compound, optionally wherein the surrogate is Iodine-124, and the radiotherapeutic compound is Iodine-131; or
wherein the biomarker indicates dosages corresponding to different uptake metrics; or
wherein the uptake metric is based on a standardized uptake value (SUV), optionally wherein the uptake metric is the SUV normalized by lean body mass (SUL); or
wherein the radiotherapeutic compound is I¹³¹, PSMA-617, lutetium Lu 177 dotatate, or radiolabeled DOTA hapten.

14. (canceled)

15. (canceled)

16. (canceled)

17. (canceled)

18. (canceled)

19. (canceled)

20. (canceled)

21. The computer-implemented method of claim 12, further comprising:
detecting, in a single-time-point medical image of a patient, for each cancerous lesion of the patient, an indicator of a surrogate of a radiotherapeutic compound, the medical image captured using the medical imaging system within a predetermined time range following administration of the surrogate to the patient;
determining, based on each indicator, an uptake metric corresponding to uptake of the surrogate by each corresponding cancerous lesion;
generating, based on the uptake metric and the predetermined time range, using the biomarker generated based on uptake metrics measured following administration of the surrogate to the cohort of subjects, a prediction of radiation dose for administration of the radiotherapeutic compound to the patient; and
providing the radiation dose prediction for subsequent use by a healthcare professional in determining a treatment protocol for treating the cancerous lesions of the patient using the radiotherapeutic compound, wherein providing the radiation dose prediction comprises at least one of storing the radiation dose prediction in a non-transitory computer-readable storage medium, outputting the radiation dose prediction using a display screen or printer for use in determining the treatment protocol, or transmitting the radiation dose prediction to another computing device or computing system for subsequent use in use in determining the treatment protocol.

22. (canceled)

23. (canceled)

24. (canceled)

25. A computer-implemented method comprising:
acquiring a medical image of a patient captured at an amount of time following administration of a surrogate of a radiotherapeutic compound to the patient, the amount of time falling within a predetermined time range following the administration of the surrogate;
determining, based on the medical image, an uptake metric corresponding to uptake of the surrogate by each cancerous lesion of the patient;
generating, based on the amount of time and the uptake metrics, using a biomarker, a radiation dose prediction for administration of the radiotherapeutic compound to the patient, the radiation dose predicted to produce an anti-tumor response, the biomarker being based on cohort uptake metrics measured in multiple-time-point medical images of each subject in a cohort of subjects following administration of the surrogate or administration of the radiotherapeutic compound to each subject in the cohort; and
outputting the radiation dose for use in determining a treatment protocol comprising using the radiotherapeutic compound to treat the cancerous lesions of the patient based on the radiation dose prediction.

26. The method of claim 25, further comprising capturing the medical image using a medical imaging system.

27. The method of claim 25, further comprising administering, at the amount of time prior to acquiring the medical image, the surrogate of the radiotherapeutic compound to the patient.

28. The method of claim 25, wherein determining the uptake metric corresponding to uptake of the surrogate by each cancerous lesion of the patient comprises detecting, in the medical image, for each cancerous lesion of the patient, an indicator of the surrogate, and generating the uptake metric based on the indicator.

29. The method of claim 23, further comprising administering the treatment to the patient.

30. A method comprising:
capturing, using a medical imaging system, a medical image of a patient at an amount of time following administration of a surrogate of a radiotherapeutic compound to the patient, the amount of time falling within a predetermined time range following the administration of the surrogate;
determining, based on the medical image, an uptake metric corresponding to uptake of the surrogate by each cancerous lesion of the patient;
generating, based on the single time point and the uptake metric, using a biomarker generated based on uptake metrics measured following administration of the surrogate or administration of the radiotherapeutic compound to a cohort of subjects, a prediction of radiation dose for administration of the radiotherapeutic compound to the patient; and
determining a treatment protocol for treating the cancerous lesions of the patient based on the prediction of radiation dose.

31. The method of claim 30, further comprising administering a treatment to the patient based on the treatment protocol.

32. The method of claim 1, wherein the patient or patients comprises a genetic mutation that regulates uptake of the surrogate.

33. The method of claim 32, wherein the genetic mutation is BRAF V600E.

34. The method of claim 1, wherein the patient or patients have received or are receiving a MEK inhibitor and/or a BRAF inhibitor.

35. A computing system comprising one or more processors that are configured to implement any of the methods of claim 1.

36. A computer-readable storage medium comprising instructions configured to cause one or more processors of a computing system to implement any of the methods of claim 1.
